(19) United States
(12) Patent Application Publication
(10) Pub. No.: US 2024/0252790 A1
(43) Pub. Date: Aug. 1, 2024

(54) CATHETER SYSTEM HAVING A SWAB FOR SITE DISINFECTION

(71) Applicant: Becton, Dickinson and Company, Franklin Lakes, NJ (US)

(72) Inventors: Shishir Prasad, Ramsey, NJ (US); Manish Kumar, Bengaluru (IN); Karthik MR, Bangalore (IN); S. Ray Isaacson, Layton, UT (US); Jonathan Karl Burkholz, Salt Lake City, UT (US)

(21) Appl. No.: 18/103,329

(22) Filed: Jan. 30, 2023

(51) Int. Cl. A61M 25/06

(52) U.S. Cl. CPC A61M 25/0618; A61M 25/0606; A61M 2207/10; A61M 2209/10

(57) ABSTRACT

A catheter system may include a catheter adapter, which may include a distal end, a proximal end, and a lumen. The catheter system may include one or more of the following: a catheter tube extending distally from the distal end of the catheter adapter; a needle hub; an introducer needle extending from the needle hub and distally through the catheter tube, wherein the introducer needle comprises a sharp distal tip; a needle cover surrounding the catheter tube and the sharp distal tip; a swab holder coupled to a distal end of the needle cover; and a swab secured within the swab holder and containing a disinfectant. an end cap proximal to the catheter adapter. The catheter system may include an end cap proximal to the catheter adapter, and the swab holder may be coupled to a swab holder coupled to a proximal end of the end cap.

### BACKGROUND

A common type of catheter assembly includes a peripheral intravenous catheter (“PIVC”) that is over-the-needle. As its name implies, the PIVC that is over-the-needle may be mounted over an introducer needle having a sharp distal tip. The catheter assembly may include a catheter adapter, the PIVC extending distally from the catheter adapter, and the introducer needle extending through the PIVC. The PIVC and the introducer needle may be assembled such that the distal tip of the introducer needle extends beyond the distal tip of the PIVC with the bevel of the needle facing up away from skin of a patient immediately prior to insertion into the skin. The PIVC and the introducer needle are generally inserted at a shallow angle through the skin into vasculature of the patient.

In order to verify proper placement of the introducer needle and/or the PIVC in the blood vessel, a clinician may confirm that there is flashback of blood in a flashback chamber of the catheter assembly. Once placement of the introducer needle has been confirmed, the clinician may remove the introducer needle, leaving the PIVC in place for future blood withdrawal or fluid infusion.

Alcohol swabbing is a common technique used to apply a disinfectant to the skin prior to cannulation or insertion of the PIVC into the vein, as well as other surgical or non-surgical procedures. It is often inconvenient for the clinician to carry individually packaged alcohol wipes because they take up additional space and may be purchased and carried in bulk. Also, individual alcohol wipes are often small and easy to misplace. Additionally, many diabetics have dexterity problems that make it difficult to open packaging containing alcohol wipes. Further, single-handed operation may not be realized when using alcohol wipes packaged separately. Moreover, when the alcohol wipe is removed from its package, some of the alcohol may remain in the package unused. Also, direct contact by the clinician with the alcohol wipe during disinfection of the skin may contaminate an insertion site.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices and related methods. More particularly, the present disclosure relates to a catheter system having a swab or wipe for site disinfection, as well as related devices and methods. In some embodiments, the catheter system may include a catheter adapter, which may include a distal end, a proximal end, and a lumen extending through the distal end of the catheter adapter and the proximal end of the catheter adapter. In some embodiments, the catheter system may include a catheter tube extending distally from the distal end of the catheter adapter.

In some embodiments, the catheter system may include a needle hub and an introducer needle extending from the needle hub and distally through the catheter tube. In some embodiments, the introducer needle may include a sharp distal tip. In some embodiments, the catheter system may include a needle cover surrounding the catheter tube and the sharp distal tip. In some embodiments, the catheter system may include a swab holder coupled to a distal end of the needle cover. In some embodiments, the catheter system may include the swab secured within the swab holder and containing a disinfectant. Thus, the swab may be integrated with the catheter system.

In some embodiments, the catheter system may include a film coupled to the swab holder and sealing the swab from an external environment. In some embodiments, the swab holder and/or the film may include a thermoplastic, and the film may thus be heat sealed to the swab holder. In some embodiments, the needle cover may be tubular.

In some embodiments, the swab may be circular or oval. In some embodiments, the swab is rectangular or square. In some embodiments, the swab may include a sponge. In some embodiments, the sponge may be precut.

In some embodiments, the swab holder may include a flange surrounding and contacting the swab. In some embodiments, the swab may extend outwardly beyond the flange. In some embodiments, the swab may be oval, and the flange may be oval. In some embodiments, the swab is rectangular, and the flange may be rectangular. In some embodiments, the needle cover may extend from the distal end of the catheter adapter beyond the sharp distal tip of the introducer needle. In some embodiments, the catheter tube may be disposed within the needle cover.

In some embodiments, the catheter system may include an end cap proximal to the catheter adapter. In some embodiments, the swab holder may be coupled to a proximal end of the end cap. In some embodiments, the swab may be secured within the swab holder and contain a disinfectant.

A method of manufacture may include bonding the swab to the swab holder configured to couple to the distal end of the needle cover or the proximal end of an end cap of the catheter system. In some embodiments, the method may include after bonding the swab to the swab holder, filling the swab with the disinfectant. In some embodiments, the method may include after filling the swab with the disinfectant, placing the film over the swab. In some embodiments, the method may include after placing the film over the swab, heat sealing the film to the swab holder.

In some embodiments, the swab holder may include the flange surrounding and contacting the swab. In some embodiments, the swab may extend outwardly beyond the flange. In some embodiments, the flange may form a pocket. In some embodiments, bonding the swab to the swab holder may include placing glue within the pocket and contacting the swab with the glue.

In some embodiments, the method may include coupling the swab holder to the distal end of the needle cover or the proximal end of the end cap. In some embodiments, the method may include after coupling the swab holder to the distal end of the needle cover, coupling the needle cover to the distal end of the catheter adapter such that the needle cover covers the introducer needle and the catheter tube. In some embodiments, the catheter system may include a connector coupled to the proximal end of the catheter adapter. In some embodiments, the method may include after coupling the swab holder to the proximal end of the end cap, coupling the end cap to the connector.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of a catheter system;

FIG. 1B is an upper perspective view of the catheter system, illustrating an example needle cover and example swab holder coupled to the needle cover;

FIG. 1C is an exploded view of the needle cover, the swab holder, an example swab, and an example film, according to some embodiments;

FIG. 2A is an upper perspective of the catheter system, illustrating an example rectangular swab and example rectangular swab holder, according to some embodiments;

FIG. 2B is a top view of the rectangular swab and the rectangular swab holder, according to some embodiments;

FIG. 2C is a side view of the rectangular swab and the rectangular swab holder, according to some embodiments;

FIG. 2D is an upper perspective view of the rectangular swab and the rectangular swab holder, illustrating the rectangular swab including a pre-cut sponge, according to some embodiments;

FIG. 3A is an upper perspective of the catheter system, illustrating an example oval swab and example oval swab holder, according to some embodiments;

FIG. 3B is a top view of the oval swab and the oval swab holder, according to some embodiments;

FIG. 3C is a side view of the oval swab and the oval swab holder, according to some embodiments;

FIG. 3D is an upper perspective view of the oval swab and the oval swab holder, illustrating the oval swab including a pre-cut sponge, according to some embodiments;

FIG. 4A is an upper perspective view of the catheter system, illustrating the swab holder coupled to an example end cap, according to some embodiments;

FIG. 4B is an upper perspective view of the catheter system, illustrating the swab holder removed from the end cap, according to some embodiments;

FIG. 4C is an upper perspective view of the catheter system, illustrating the swab holder and the end cap removed, according to some embodiments; and

FIG. 4D is a cross-sectional view of the catheter system, illustrating the swab holder coupled to the end cap, according to some embodiments.

### DESCRIPTION OF EMBODIMENTS

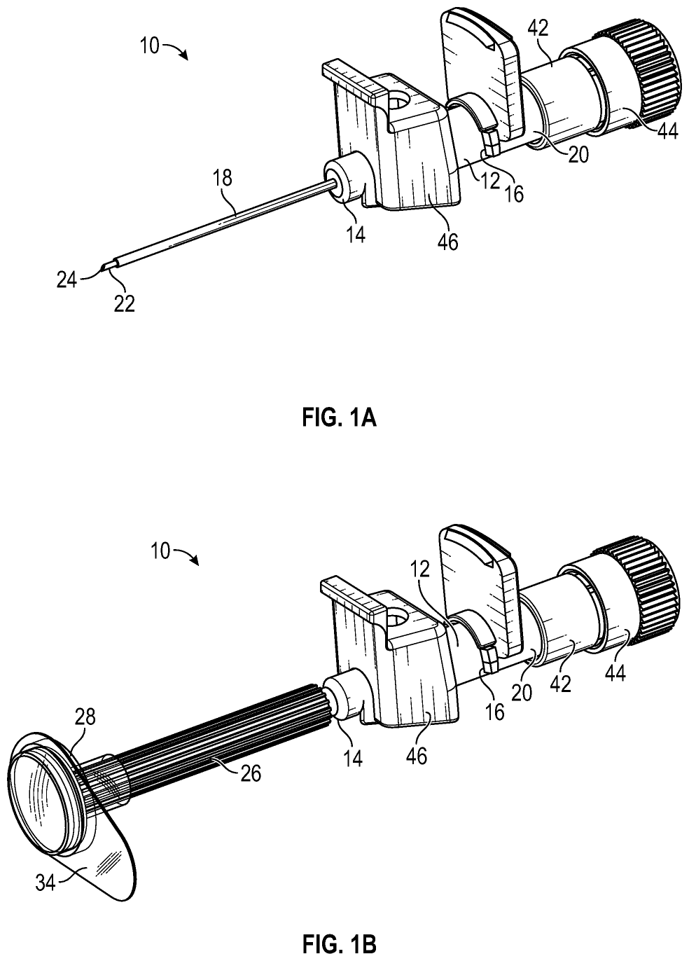

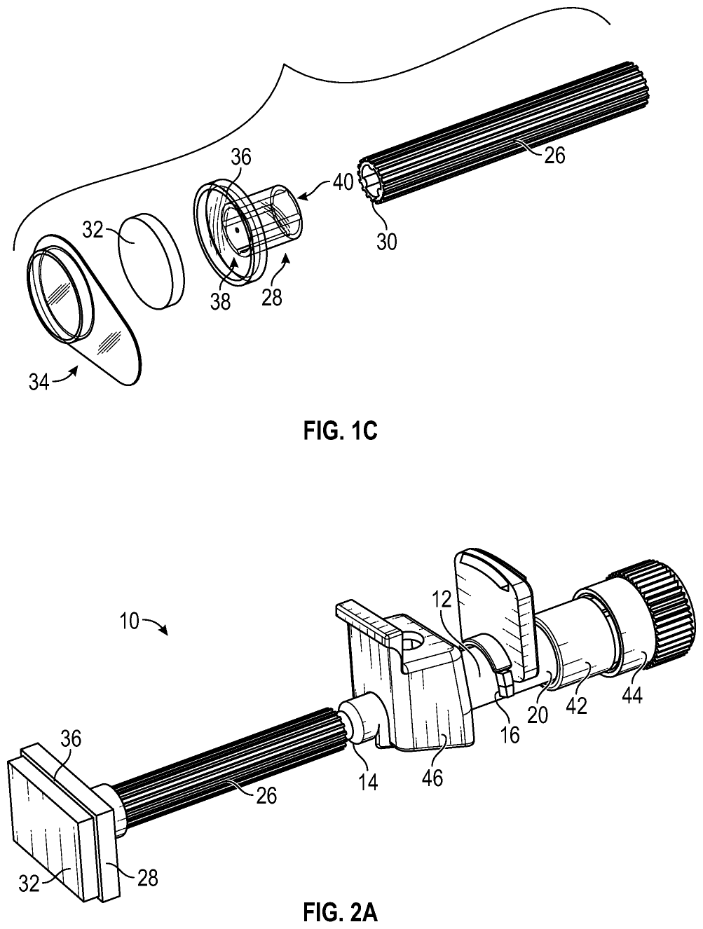

Referring now to FIGS. 1A-1C, in some embodiments, a catheter system 10 may include a catheter adapter 12, which may include a distal end 14, a proximal end 16, and a lumen extending through the distal end 14 of the catheter adapter 12 and the proximal end 16 of the catheter adapter 12. In some embodiments, the catheter system 10 may include a catheter tube 18 extending distally from the distal end 14 of the catheter adapter 12. In some embodiments, the catheter system 10 may include a peripheral intravenous catheter system, a midline catheter system, an arterial catheter system, a peripherally-inserted central catheter system, or another suitable catheter system.

In some embodiments, the catheter system 10 may include a needle hub 20 and an introducer needle 22 extending from the needle hub 20 and distally through the catheter tube 18. In some embodiments, the introducer needle 22 may include a sharp distal tip 24 configured to pierce skin and vasculature of the patient to facilitate placement of the catheter tube 18 within the vasculature. In some embodiments, the catheter system 10 may include a needle cover 26 surrounding the catheter tube 18 and the sharp distal tip 24. In some embodiments, the needle cover 26 may be tubular. In some embodiments, the needle cover 26 may extend from the distal end 14 of the catheter adapter 12 beyond the sharp distal tip 24. In some embodiments, the catheter tube 18 may be disposed within the needle cover 26.

In some embodiments, the catheter system 10 may include a swab holder 28 coupled to a distal end 30 of the needle cover 26. In some embodiments, the catheter system 10 may include a swab 32 secured within the swab holder 28 and containing a disinfectant. In some embodiments, the disinfectant may include isopropyl alcohol (IPA), such as, for example, 70% IPA. In some embodiments, the swab 32 may contain another suitable disinfectant. In some embodiments, the swab 32 may be used to disinfect a site on the skin of the patient, such as a catheter system insertion site, before insertion of the catheter tube 18 and the introducer needle 22 at the site.

In some embodiments, the swab 32 may include a non-woven material, such as paper or another suitable material configured to hold the disinfectant. In some embodiments, the swab 32 may include a woven fabric. In some embodiments, the swab 32 may include a sponge, which may be porous and absorbent. In some embodiments, the swab holder 28 may hold the disinfectant by absorption and/or may be soaked in the disinfectant. In some embodiments, touching and/or compression of the swab 32 against the skin may release the disinfectant onto the skin.

In some embodiments, the catheter system 10 may include a film 34 coupled to the swab holder 28 and maintaining the sterility of the swab 32 until usage. In these embodiments, the film 34 may seal the swab 32 from an external environment to prevent drying out and/or bacterial contamination of the swab 32. In some embodiments, the film 34 may include thermoplastic. In some embodiments, the swab holder 28 may include a thermoplastic. In some embodiments, the film 34 and/or the swab holder 28 including thermoplastic may facilitate the film 34 being heat sealed to the swab holder 28. In some embodiments, the film 34 and the swab holder 28 may be sealed together by another suitable method, such as, for example, hot melt or pressure-sensitive adhesive.

In some embodiments, the swab holder 28 may include a flange 36 surrounding and contacting the swab 32. In some embodiments, the swab 32 may extend outwardly beyond the flange 36, which may facilitate contact of the swab 32 with the skin for disinfection. In some embodiments, the flange 36 may form a pocket 38 in which the swab 32 may be seated. In some embodiments, the distal end 30 of the needle cover 26 may be inserted within a proximal opening 40 of the swab holder 28 and secured via an interference or press fit, adhesive, or another suitable coupling mechanism. In some embodiments, coupling the needle cover 26 and the swab holder 28 together by inserting the distal end 30 of the needle cover 26 into the proximal opening 40 may prevent the swab holder 28 from interfering with and/or contacting the introducer needle 22 and the catheter tube 18 within the needle cover 26. In some embodiments, a diameter of the pocket 38 may be greater than a diameter of the proximal opening 40 to facilitate use of a swab 32 having an increased surface area to facilitate disinfecting the site. In some embodiments, a bottom of the pocket 38 may be solid to provide a barrier.

In some embodiments, the clinician may remove the film 34 from the swab holder 28, and then the clinician may hold the catheter system 10 to swab and disinfect the site. In some embodiments, the catheter system 10 may provide ergonomic comfort to the clinician compared to conventional methods, such as using wipes. In further detail, in some embodiments, the needle cover 26 may act like a handle, which the clinician may hold like a pencil, providing good stability and usability to the clinician for effective swabbing. In these and other embodiments, an outer surface of the needle cover 26 may be cylindrical. In some embodiments, following swabbing and disinfection of the site, the clinician may remove the needle cover 26 and insert the catheter tube 18 into the vasculature, aided by the introducer needle 22 extending through the catheter tube 18.

In some embodiments, an adapter or connector 42 may be coupled to a proximal end of the needle hub 20 and/or an end cap 44 may be coupled to a proximal end of the connector 42. In some embodiments, the connector 42 may connect the needle hub 20 to the end cap 44. In some embodiments, the connector 42 may include a vent plug configured to pass air but not blood. In some embodiments, the end cap 44 may be coupled directly to the needle hub 20. In some embodiments, the end cap 44 may be configured to close a proximal end of the catheter system 10 and may not pass air or liquid. In some embodiments, the catheter system 10 may include a housing 46, which may be configured to hold one or more folded wings extending from the catheter adapter 12. In some embodiments, a configuration of the catheter system 10 may vary. For example, the catheter system 10 may not include the housing 46. In some embodiments, the catheter system 10 may include another configuration suitable for a peripheral intravenous catheter system, a midline catheter system, an arterial catheter system, a peripherally-inserted central catheter system, or another suitable catheter system.

In some embodiments, integrating the swab 32 in the catheter system 10 may enhance clinician compliance with skin preparation protocols before cannulation, which are many times not complied with due to a cleaning agent not being available at the bedside. In some embodiments, integrating the swab 32 also decreases a length of a procedure because it is not necessary for the clinician to search for a cleaning agent. Moreover, in some embodiments, the catheter system 10 can be used like a marker or applicator after the swab 32 is exposed, which facilitates an aseptic non-touch technique in which neither the swab 32 nor the disinfectant is contacted by the clinician.

In some embodiments, the swab 32 may be circular or oval, as illustrated, for example, in FIGS. 1A-1C. In some embodiments, the swab 32 that is circular or oval may reduce a number of strokes needed to disinfect the site. In further detail, a particular swab that is rectangular or square may require a horizontal and a vertical stroke to cover a same surface area as the swab 32 that is circular or oval.

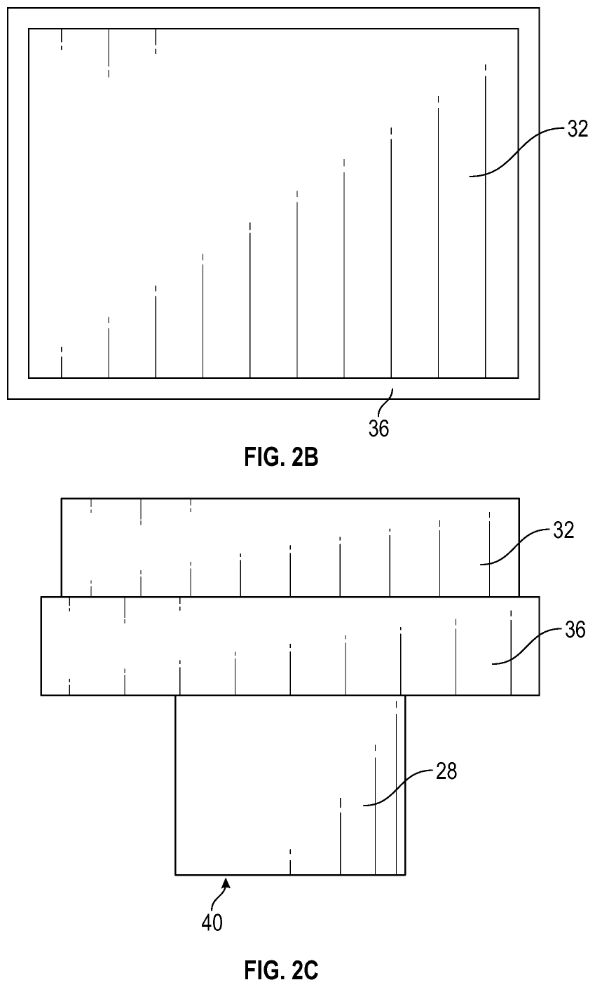

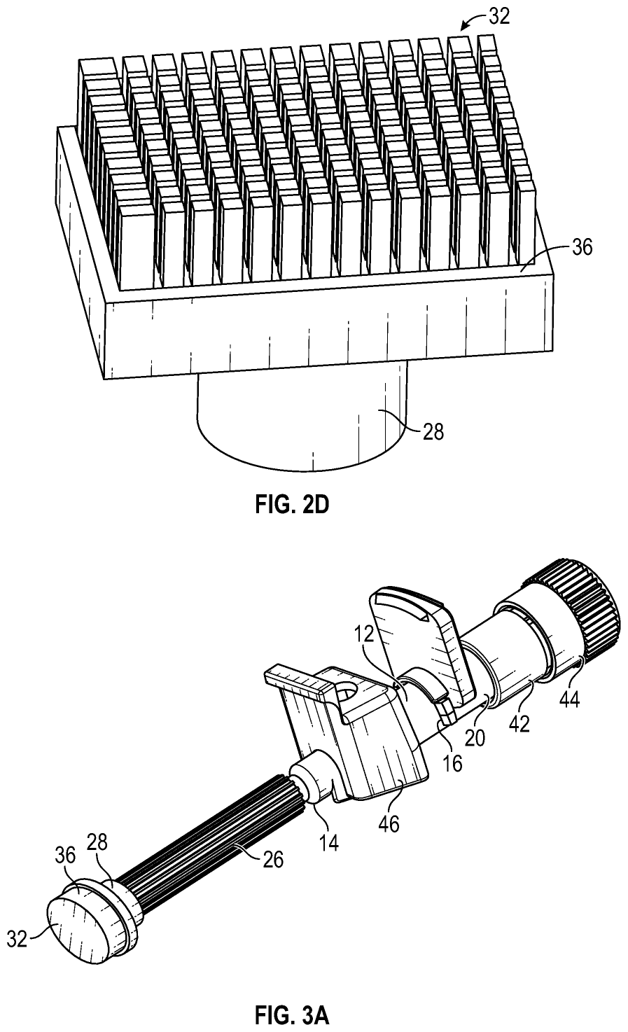

Referring now to FIGS. 2A-2D, in some embodiments, the swab 32 may be rectangular or square. In some embodiments, the swab 32 is rectangular, and the flange 36 may be rectangular to fit around the swab 32. As illustrated, for example, in FIG. 2C, in some embodiments, the swab 32 may extend outwardly beyond the flange 36, which may facilitate contact of the swab 32 with the skin for disinfection. As illustrated, for example, in FIG. 2D, in some embodiments, the swab 32 may include a sponge, which may be precut. In some embodiments, the swab 32 that is precut increases a surface area of the sponge such that the sponge is able to hold a greater volume of the disinfectant. In some embodiments, the sponge that is precut may include multiple arms which may facilitate active scrubbing of the site for improved disinfection. In some embodiments, active scrubbing may include disinfection due to a combined effect of mechanical removal of microbes due to friction-assisted wiping and chemical disinfection due to the disinfectant in the swab 32.

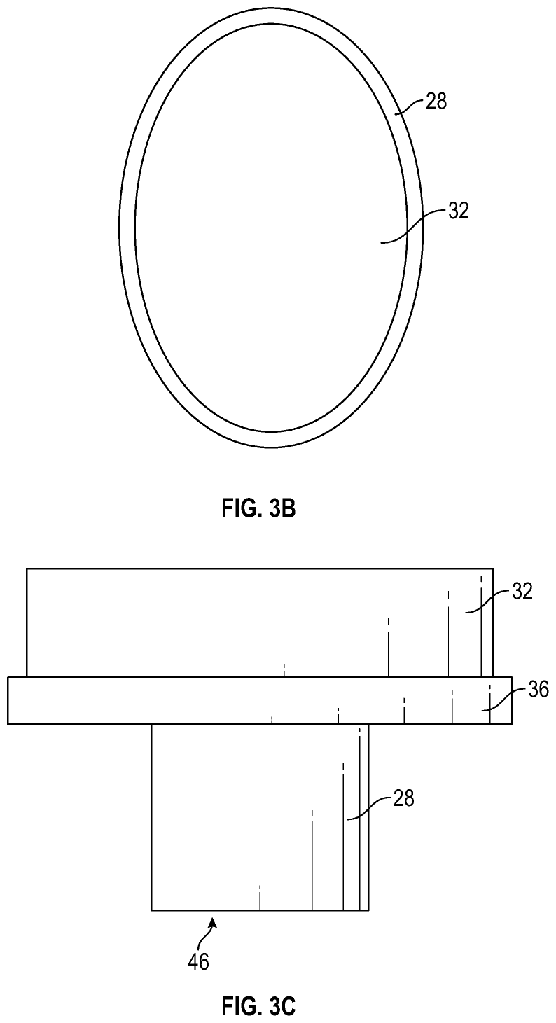

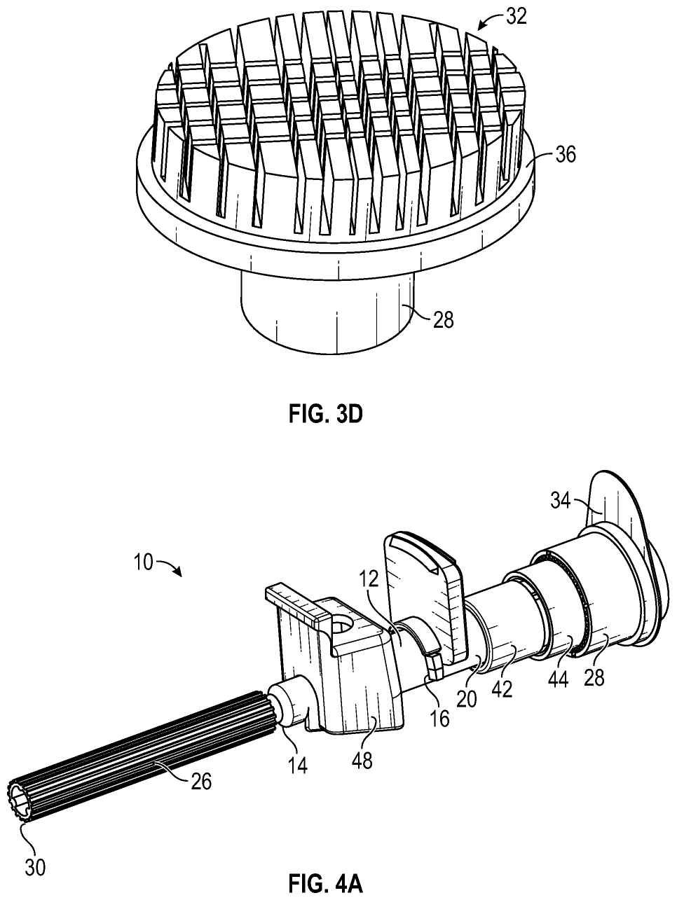

Referring now to FIGS. 3A-3D, in some embodiments, the swab 32 may be oval and/or the flange 36 may be oval to fit around the swab 32. As illustrated, for example, in FIG. 3D, in some embodiments, the swab 32 may be precut. In some embodiments, the swab 32 that is precut increases a surface area of the sponge such that the sponge is able to hold a greater volume of the disinfectant. In some embodiments, the sponge that is precut may include multiple arms which may facilitate active scrubbing of the site for improved disinfection.

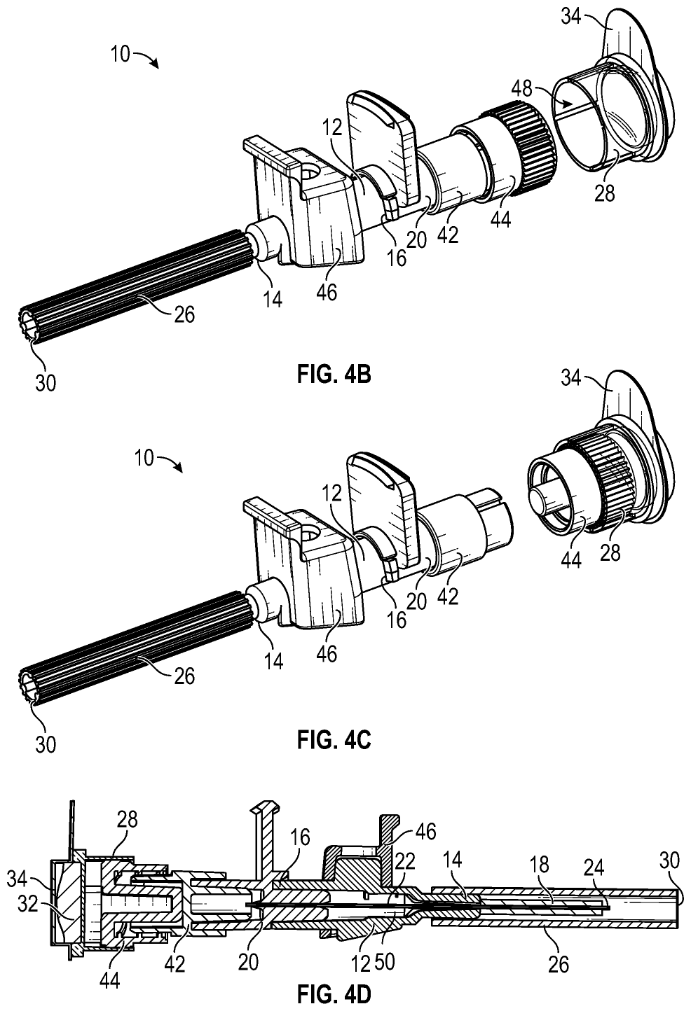

Referring now to FIGS. 4A-4D, in some embodiments, the catheter system 10 may include the end cap 44 proximal to the catheter adapter 12. In some embodiments, the swab holder 28 may be coupled to a proximal end of the end cap 44. In some embodiments, a distal end of the end cap 44 may include a luer, such as, for example, a male luer. In some embodiments, the swab holder 28 may be disposed at a proximal end or rear of the catheter system 10, which may facilitate easy access to the swab 32.

In some embodiments, the swab holder 28 may include a distal opening 48 at a distal end of the swab holder 28, and the end cap 44 may be configured to insert within the distal opening 48 and be secured therein in an interference or press fit. In some embodiments, after swabbing the site with the swab 32, the end cap 44 and the swab holder 28 may be removed from the catheter system 10 together, as illustrated, for example, in FIG. 4C, and/or discarded. In these embodiments, a remaining portion of the catheter system 10 can then be inserted into vasculature of the patient. A lumen 50 of the catheter adapter 12 with the introducer needle 22 extending therethrough is illustrated, according to some embodiments.

Referring now to FIGS. 1-4, a method of manufacture may include bonding the swab 32 to the swab holder 28 configured to couple to the distal end 30 of the needle cover 26 or the proximal end of the end cap 44 of the catheter system 10. In some embodiments, the method may include after bonding the swab 32 to the swab holder 28, filling the swab 32 with the disinfectant. In some embodiments, the method may include after filling the swab 32 with the disinfectant, placing the film 34 over the swab 32. In some embodiments, the method may include after placing the film 34 over the swab 32, heat or pressure sealing the film 34 to the swab holder 28. Heat or pressure sealing the film 34 to the swab holder 28 may ensure the disinfectant is not wasted.

In some embodiments, the swab holder 28 may include the flange 36 surrounding and contacting the swab 32. In some embodiments, the swab 32 may extend outwardly beyond the flange 36. In some embodiments, the flange 36 may form the pocket 38 within the flange 36. In some embodiments, bonding the swab 32 to the swab holder 28 may include placing glue within the pocket 38 and contacting the swab 32 with the glue.

In some embodiments, the method may include coupling the swab holder 28 to the distal end of the needle cover 26 or the proximal end of the end cap 44. In some embodiments, the method may include after coupling the swab holder 28 to the distal end 30 of the needle cover 26, coupling the needle cover 26 to the distal end 14 of the catheter adapter 12 such that the needle cover 26 covers the introducer needle 22 and the catheter tube 18. In some embodiments, the catheter system 10 may include the connector 42 coupled to the proximal end of the needle hub 20. In some embodiments, the method may include after coupling the swab holder 28 to the proximal end of the end cap 44, coupling the end cap 44 to the connector 42.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter system, comprising:
a catheter adapter, comprising a distal end, a proximal end, and a lumen extending through the distal end of the catheter adapter and the proximal end of the catheter adapter;
a catheter tube extending distally from the distal end of the catheter adapter;
a needle hub;
an introducer needle extending from the needle hub and distally through the catheter tube, wherein the introducer needle comprises a sharp distal tip;
a needle cover surrounding the catheter tube and the sharp distal tip;
a swab holder coupled to a distal end of the needle cover; and
a swab secured within the swab holder and containing a disinfectant.

2. The catheter system of claim 1, further comprising a film coupled to the swab holder and sealing the swab from an external environment.

3. The catheter system of claim 2, wherein the swab holder or the film comprises a thermoplastic or pressure sensitive material, wherein the film is heat or pressure sealed to the swab holder.

4. The catheter system of claim 1, wherein the needle cover is tubular.

5. The catheter system of claim 1, wherein the swab is circular or oval.

6. The catheter system of claim 1, wherein the swab is rectangular or square.

7. The catheter system of claim 1, wherein the swab comprises a sponge.

8. The catheter system of claim 7, wherein the sponge is precut.

9. The catheter system of claim 1, wherein the swab holder comprises a flange surrounding and contacting the swab, wherein the swab extends outwardly beyond the flange.

10. The catheter system of claim 9, wherein the swab is oval, wherein the flange is oval.

11. The catheter system of claim 9, wherein the swab is rectangular, wherein the flange is rectangular.

12. The catheter system of claim 1, wherein the needle cover extends from the distal end of the catheter adapter beyond the sharp distal tip of the introducer needle, wherein the catheter tube is disposed within the needle cover.

13. A catheter system, comprising:
a catheter adapter, comprising a distal end, a proximal end, and a lumen extending through the distal end of the catheter adapter and the proximal end of the catheter adapter;
a catheter tube extending distally from the distal end of the catheter adapter;
a needle hub;
an introducer needle extending from the needle hub and distally through the catheter tube, wherein the introducer needle comprises a sharp distal tip;
an end cap proximal to the catheter adapter;
a swab holder coupled to a proximal end of the end cap; and
a swab secured within the swab holder and containing a disinfectant.

14. The catheter system of claim 13, further comprising a film coupled to the swab holder and sealing the swab from an external environment.

15. The catheter system of claim 13, wherein the swab holder or the film comprises a thermoplastic or pressure sensitive material, wherein the film is heat or pressure sealed to the swab holder.

16. The catheter system of claim 13, wherein the swab holder comprises a flange surrounding and contacting the swab, wherein the swab extends outwardly beyond the flange.

17. A method of manufacture, comprising:
bonding a swab to a swab holder configured to couple to a distal end of a needle cover of a catheter system or a proximal end of an end cap of the catheter system;
after bonding the swab to the swab holder, filling the swab with a disinfectant;
after filling the swab with the disinfectant, placing a film over the swab; and
after placing the film over the swab, heat or pressure sealing the film to the swab holder.

18. The method of claim 17, wherein the swab holder comprises a flange surrounding and contacting the swab, wherein the swab extends outwardly beyond the flange, wherein the flange forms a pocket, wherein bonding the swab to the swab holder comprises placing glue within the pocket and contacting the swab with the glue.

19. The method of claim 17, wherein the catheter system comprises:
a catheter adapter, comprising a distal end, a proximal end, and a lumen extending through the distal end of the catheter adapter and the proximal end of the catheter adapter;
a catheter tube extending distally from the distal end of the catheter adapter;
a needle hub; and
an introducer needle extending from the needle hub and distally through the catheter tube, wherein the introducer needle comprises a sharp distal tip;
further comprising coupling the swab holder to the distal end of the needle cover, and after coupling the swab holder to the distal end of the needle cover, further comprising coupling the needle cover to the distal end of the catheter adapter such that the needle cover covers the introducer needle and the catheter tube.

20. The method of claim 17, wherein the catheter system comprises:
a catheter adapter, comprising a distal end, a proximal end, and a lumen extending through the distal end of the catheter adapter and the proximal end of the catheter adapter;
a catheter tube extending distally from the distal end of the catheter adapter;
a needle hub;
an introducer needle extending from the needle hub and distally through the catheter tube, wherein the introducer needle comprises a sharp distal tip;
an end cap proximal to the catheter adapter; and
a connector coupled to a proximal end of the needle hub;
further comprising after coupling the swab holder to the proximal end of the end cap, coupling the end cap to the connector.
